(19) United States
(12) Patent Application Publication
(10) Pub. No.: US 2017/0301050 A1
(43) Pub. Date: Oct. 19, 2017

(54) SYSTEM AND METHOD OF ANALYZING HEALTH CARE PROVIDER NETWORKS

(71) Applicant: MEDICAL KNOWLEDGE GROUP LLC, New York, NY (US)

(72) Inventor: Leon Behar, Scarsdale, NY (US)

(21) Appl. No.: 15/099,322

(22) Filed: Apr. 14, 2016

(51) Int. Cl. G06Q 50/22; G06F 17/30

(52) U.S. Cl. CPC G06Q 50/22; G06F 17/3053; G06F 17/30867

(57) ABSTRACT

A computer system and method for creating a network model of health care providers comprising individuals that are connected to a particular influencer or health care provider. The system determines an influence rank that is assigned to each network that corresponds to the level of influence in comparison to other networks in a cohort. For example, the influence rank could be based, at least in part, on the number of patients treated by a health care provider and that provider's patient referrals. In some cases, the system provides a graphical user interface through which the networks can be visualized. These networks can be filtered based on a variety of categories to pinpoint specific networks, such as by geographic regions, specialties, diseases, and/or specific health care providers.

### TECHNICAL FIELD

This disclosure relates generally to computer systems and methods for detecting networks among health care providers; in particular, this disclosure relates to a computerized system and method in which claims data can be analyzed to identify, among other things, networks among and relative influences of health care providers in their community.

### BACKGROUND AND SUMMARY

In the medical field, there are certain health care providers that are influential and can impact clinical practice behavior locally, regionally and nationally. These influencers can have a disproportionate impact on the sales volumes and product adoption of health care products and services. However, the identification of such influencers can be challenging and time consuming. Moreover, it can be easy to overlook those health care providers that are truly influential.

This disclosure relates to a computerized system and method for creating a network model of health care providers comprising individuals that are connected to a particular influencer or health care provider. In some embodiments, the system determines an influence rank that is assigned to each network that corresponds to the level of influence in comparison to other networks in a cohort. For example, the influence rank could be based, at least in part, on the number of patients treated by a health care provider and that provider's patient referrals. In some cases, the system provides a graphical user interface through which the networks can be visualized. These networks can be filtered based on a variety of categories to pinpoint specific networks, such as by geographic regions, specialties, diseases, and/or specific health care providers. Typically, the visualization graphically illustrates a relative amount of influence by each health care provider in a network so top influencers can be easily identified. For example, a size, shape, color and/or other identifier could be used to show relative influence.

According to one aspect, this disclosure provides an apparatus with a storage device and at least one processor coupled to the storage device. The storage device stores a program for controlling the at least one processor and the program causes the processor to obtain historical claims data representative of encounters between a plurality of patients and health care providers (“HCPs”), including referrals between HCPs. The processor analyzes the historical claims data to create a network model representative of connections between HCPs and determines an influence score for the HCPs that represents a level of influence of a respective HCP in comparison to other HCPs that are connected to that respective HCP. Data representative of at least a portion of the network model, including the influence score of those HCPs in that portion of the network, is then transmitted by the processor. In some embodiments, at least a portion of the connections in the network model are based on referrals between HCPs. There are a variety of potential factors for the influence score, such as a number of patients treated directly by a HCP, patients of a selected HCP treated by other HCPs who are directly influenced by the selected HCP, patients of a selected HCP treated by other HCPs who are directly or indirectly influenced by the selected HCP, a sum of patients treated and patients indirectly influenced by a selected HCP, a number of HCPs influenced by a selected HCP either directly or indirectly regardless of how many patients treated by the selected HCP.

In some embodiments, the program is configured to cause the processor to generate an interface from which the network model representing connections between HCPs can be viewed. For example, the interface may include a data table representative of one or more criteria that comprise the network model. In some cases, the interface includes a graphical visualization of the network model. For example, the graphical visualization could include one or more nodes representative of respective HCP with connections between the nodes representing connected HCPs in the network model. The graphical visualization could be configured to visually differentiate nodes based on relative influence, such as by relative size and/or color of the nodes. In some embodiments, the interface could include one or more filters configured to remove information about the network model from the interface based on user-selected filter criteria, such as geographic regions, specialties, diseases, and/or specific health care providers.

According to another aspect, this disclosure provides a computer-implemented method. The method includes the step of obtaining historical claims data representative of encounters between a plurality of patients and health care providers, including referrals between HCPs. The historical claims data is analyzed to create a network model representative of connections between HCPs. An influence score is determined for the HCPs that represents a level of influence of a respective HCP in comparison to other HCPs that are connected to that respective HCP. Next, the method transmits data representative of at least a portion of the network model with the influence score of those HCPs in that portion of the network.

According to a further aspect, this disclosure provides a tangible, non-transitory computer readable medium storing instructions that, when executed by at least one processor, causes the at least one processor to perform a method in which historical claims data representative of encounters between a plurality of patients and health care providers, including referrals between HCPs, is obtained. This claims data is analyzed to create a network model representative of connections between HCPs. An influence score for the HCPs is determined that represents a level of influence of a respective HCP in comparison to other HCPs that are connected to that respective HCP. The data representative of at least a portion of the network model with the influence score of those HCPs in that portion of the network is then transmitted.

Additional features and advantages of the invention will become apparent to those skilled in the art upon consideration of the following detailed description of the illustrated embodiment exemplifying the best mode of carrying out the invention as presently perceived. It is intended that all such additional features and advantages be included within this description and be within the scope of the invention.

### BRIEF DESCRIPTION OF THE DRAWINGS

The present disclosure will be described hereafter with reference to the attached drawings which are given as non-limiting examples only, in which:

FIG. 1 is a diagrammatic view of an example computing device in which the analysis system could operate according to one embodiment;

FIG. 2 is a diagrammatic view of an example computing environment in which the analyses system could operate according to one embodiment;

FIG. 3 is a simplified block diagraph illustrating an indirect influence;

FIG. 4 is a flow chart showing example operations of the analysis system according to one embodiment; and

FIGS. 5-22 are screen shots of an example interface for the analysis system according to one embodiment.

Corresponding reference characters indicate corresponding parts throughout the several views. The components in the figures are not necessarily to scale, emphasis instead being placed upon illustrating the principals of the invention. The exemplification set out herein illustrates embodiments of the invention, and such exemplification is not to be construed as limiting the scope of the invention in any manner.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the disclosure.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

This disclosure relates generally to a computer system and method for creating a network model of health care providers comprising individuals that are connected to a particular influencer or health care provider. In some embodiments, the system determines an influence rank that is assigned to each network that corresponds to the level of influence in comparison to other networks in a cohort. For example, the influence rank could be based, at least in part, on the number of patients treated by a health care provider and that provider's patient referrals. In some cases, the system provides a graphical user interface through which the networks can be visualized. These networks can be filtered based on a variety of categories to pinpoint specific networks, such as by geographic regions, specialties, diseases, and/or specific health care providers. Typically, the visualization graphically illustrates a relative amount of influence by each health care provider in a network so top influencers can be easily identified.

The detailed description which follows is presented in part in terms of algorithms and symbolic representations of operations on data bits within a computer memory representing alphanumeric characters or other information. An algorithm is provided by this disclosure and is generally conceived to be a self-consistent sequence of steps leading to a desired result. These steps are those requiring physical manipulations of physical quantities. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed. It should be borne in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through sophisticated software systems. Data structures are not the information content of a memory, rather they represent specific electronic structural elements which impart or manifest a physical organization on the information stored in memory. More than mere abstraction, the data structures are specific electrical or magnetic structural elements in memory which simultaneously represent complex data accurately, often data modeling physical characteristics of related items, and provide increased efficiency in computer operation.

Further, the manipulations performed are often referred to in terms, such as comparing or adding, commonly associated with mental operations performed by a human operator. No such capability of a human operator is necessary, or desirable in most cases, in any of the operations described herein which form part of the present invention; the operations are machine operations. Useful machines for performing the operations of the present invention include general purpose digital computers or other similar devices. In all cases the distinction between the method operations in operating a computer and the method of computation itself should be recognized. A method and apparatus are disclosed for operating a computer in processing electrical or other (e.g., mechanical, chemical) physical signals to generate other desired physical manifestations or signals. The computer operates on software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level coding of the instructions that is interpreted to obtain the actual computer code. The software module may also include a hardware component, wherein some aspects of the algorithm are performed by the circuitry itself, rather as a result of an instruction.

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

An apparatus is disclosed for performing these operations. This apparatus may be specifically constructed for the required purposes, or it may comprise a general purpose computer as selectively activated or reconfigured by a computer program stored in the computer. The algorithms presented herein are not inherently related to any particular computer or other apparatus unless explicitly indicated as requiring particular hardware. In some cases, the computer programs may communicate or relate to other programs or equipment through signals configured to particular protocols which may or may not require specific hardware or programming to interact. In particular, various general purpose machines may be used with programs written in accordance with the teachings herein, or it may prove more convenient to construct more specialized apparatus to perform the required method steps. The required structure for a variety of these machines will appear from the description below.

In the following description several terms which are used frequently have specialized meanings in the present context. The term “network” means two or more computers which are connected in such a manner that messages may be transmitted between the computers. In such computer networks, typically one or more computers operate as a “server,” a computer with large storage devices such as hard disk drives and communication hardware to operate peripheral devices such as printers or modems. The term “browser” refers to a program which is not necessarily apparent to the user, but which is responsible for transmitting messages between the user's computer and the network server and for displaying and interacting with network resources.

Browsers are designed to utilize a communications protocol for transmission of text and graphic information over a worldwide network of computers, namely the “World Wide Web” or simply the “Web.” Examples of browsers compatible with the present invention include the Internet Explorer browser program offered by Microsoft Corporation (Internet Explorer is a trademark of Microsoft Corporation), the Chrome browser program offered by Google Inc. (Chrome is a trademark of Google Inc.), the Safari browser program offered by Apple Inc. (Safari is a trademark of Apple Inc.) or the Firefox browser program distributed by the Mozilla Foundation (Firefox is a registered trademark of the Mozilla Foundation). The browser could operate on a desktop operating system, such as Windows by Microsoft Corporation (Windows is a trademark of Microsoft Corporation) or OS X by Apple Inc. (OS X is a trademark of Apple Inc.). In some cases, the browser could operate on mobile operating systems, such as iOS by Apple Inc. (iOS is a trademark of Apple Inc.) or Android by Google Inc. (Android is a trademark of Google Inc.). Browsers display information which is formatted in a Standard Generalized Markup Language (“SGML”) or a Hyper Text Markup Language (“HTML”), both being scripting languages which embed non-visual codes in a text document through the use of special ASCII text codes. Files in these formats may be easily transmitted across computer networks, including global information networks like the Internet, and allow the browsers to display text, images, and play audio and video recordings.

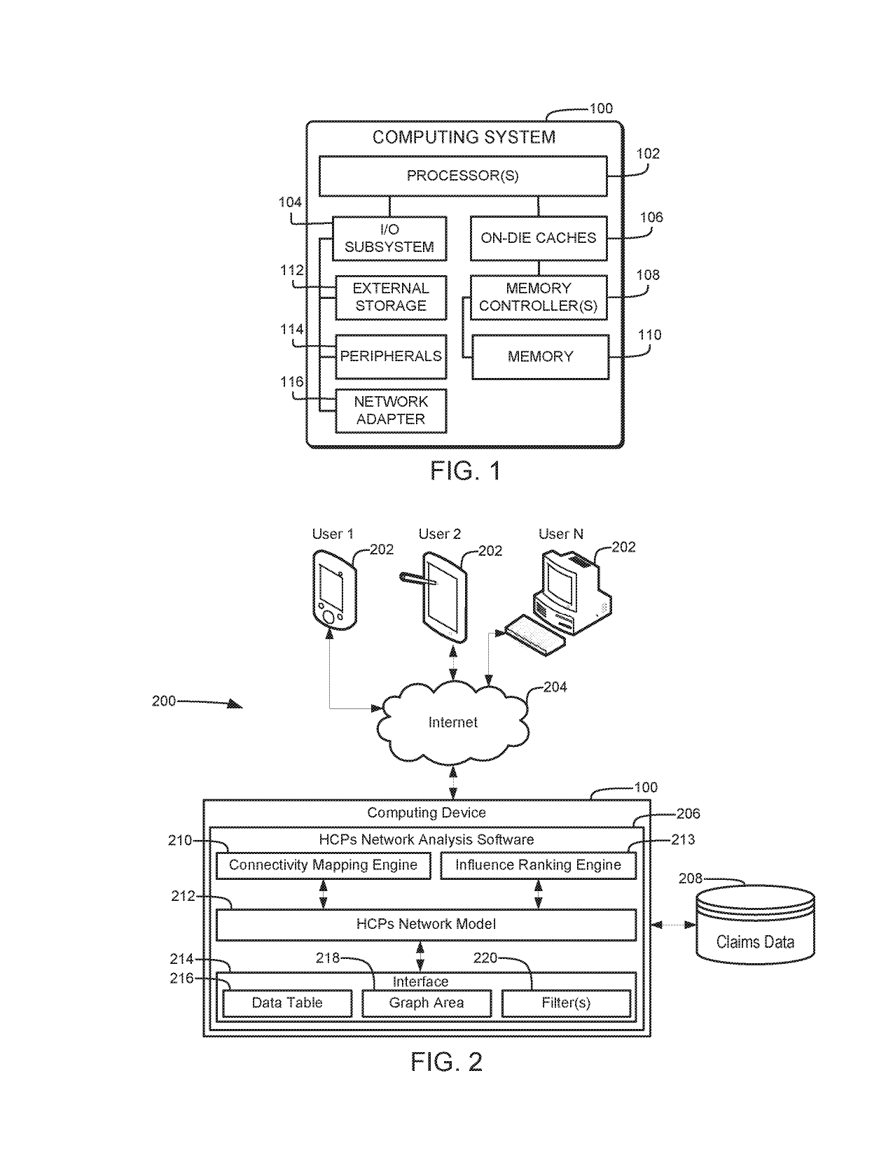

Referring now to FIG. 1, an illustrative computing device 100 for creating a network model of health care providers and assigning an influence rank, includes at least one processor 102, an I/O subsystem 104, at least one on-die cache 106, and a memory controller 108 to control a memory 110. The computing device 100 may be embodied as any type of device capable of performing the functions described herein. For example, the computing device 100 may be embodied as, without limitation, a computer, a workstation, a server computer, a laptop computer, a notebook computer, a tablet computer, a smartphone, a mobile computing device, a desktop computer, a distributed computing system, a multiprocessor system, a consumer electronic device, a smart appliance, and/or any other computing device capable of analyzing software code segments.

As shown in FIG. 1, the illustrative computing device 100 includes the processor 102, the I/O subsystem 104, the on-die cache 106, and the memory controller 108 to control a memory 110. Of course, the computing device 100 may include other or additional components, such as those commonly found in a workstation (e.g., various input/output devices), in other embodiments. For example, the computing device 100 may include an external storage 112, peripherals 114, and/or a network adapter 116. Additionally, in some embodiments, one or more of the illustrative components may be incorporated in, or otherwise form a portion of, another component. For example, the memory 110 or portions thereof, may be incorporated in the processor 102 in some embodiments.

The processor 102 may be embodied as any type of processor capable of performing the functions described herein. For example, the processor may be embodied as a single or multi-core processor(s), digital signal processor, microcontroller, or other processor or processing/controlling circuit. The memory 110 may be embodied as any type of volatile memory and/or persistent memory capable of performing the functions described herein. In operation, the memory 110 may store various data and software used during operation of the computing device 100 such as operating systems, applications, programs, libraries, and drivers. The memory 110 is communicatively coupled to the processor 102 via the memory bus using memory controller(s) 108, which may be embodied as circuitry and/or components to facilitate input/output operations with the processor 102, the memory 110, and other components of the computing device 100.

The I/O subsystem 104 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 104 may form a portion of a system-on-a-chip (SoC) and be incorporated, along with the processor 102, the memory 110, and other components of the computing device 100, on a single integrated circuit chip.

An external storage device 112 is coupled to the processor 102 with the I/O subsystem 104. The external storage device 112 may be embodied as any type of device or devices configured for short-term or long-term storage of data such as, for example, memory devices and circuits, memory cards, hard disk drives, solid-state drives, or other data storage devices.

The computing device 100 may include peripherals 114. The peripherals 114 may include any number of additional input/output devices, interface devices, and/or other peripheral devices. By way of example only, a peripheral may be a display that could be embodied as any type of display capable of displaying digital information such as a liquid crystal display (LCD), a light emitting diode (LED), a plasma display, a cathode ray tube (CRT), or other type of display device.

The computing device 100 illustratively includes a network adapter 116, which may be embodied as any communication circuit, device, or collection thereof, capable of enabling communications between the computing device 100 and other remote devices over a computer network (not shown). The network adapter 116 may be configured to use any one or more communication technology (e.g., wired or wireless communications) and associated protocols (e.g., Ethernet, Bluetooth®, Wi-Fi®, WiMAX, etc.) to effect such communication.

FIG. 2 is a high-level block diagram of a computing environment 200 under which the computing device 100 could operate according to one embodiment. FIG. 2 illustrates the computing device 100 and three clients 202 connected by a network 204. Only three clients 202 are shown in FIG. 2 in order to simplify and clarify the description. Likewise, a single computing device 100 is shown for purposes of simplicity, but multiple computing devices could be used. Embodiments of the computing environment 200 may have thousands or millions of clients 202 connected to the network 204, for example, the Internet. Users (not shown) may operate software, such as a browser, on clients 202 to both send and receive messages over network 204 via computing device 100 and its associated communications equipment and software (not shown). For example, health care providers (“HCPs”) network analysis software 206 could be accessed via computing device 100 using a browser. Typically, clients 202 would be able to access the HCPs network analysis software 206 over the network 204 by entering a web address, such as an IP address, URL, or domain name (web address generally referred to as a “Destination”) into browser software. In some embodiments, clients 202 could include a dedicated application that connects with the HCPs network analysis software 206 instead of using a web browser.

The example in FIG. 2 shows an example claims database 208 to which the HCPs network analysis software 206 has access. The claims database 208 includes historical data relating to submissions from a health care provider to an insurance company or other payor entity, such as a governmental agency, that includes information regarding encounters between health care providers and patients. This data may include, among other things, information regarding the HCP and patient, along with one or more codes describing the encounter, such as Current Procedural Terminology (“CPT”) codes and International Classification of Disease (“ICD”) codes, and National Drug Codes (“NDC”), and/or physician referral information.

As shown, the HCPs network analysis software 206 includes a connectivity mapping engine 210 that is configured to analyze historical claims data in the claims database 208 to determine a connectivity map between HCPs and patients. For example, connections between a HCP and patients could be based on information that a HCP is treating a patient, which allows a determination of a number of patients treated by HCPs. The connections between HCPs is based on referrals made between HCPs. In some embodiments, the historical claims database 208 provides information that allows the connectivity mapping engine 210 to define nodes representing each of the HCPs and edges or connections between at least a portion of HCPs based on referrals therebetween. This analysis allows the connectivity mapping engine 210 to aid in generation of a HCPs network model 212, which represents the HCPs networks. Each of these networks in the HCPs network model 212 represents individuals (patients and HCPs) that are connected to a particular influencer or HCP. In some embodiments, the network may be named after that influencer or HCP.

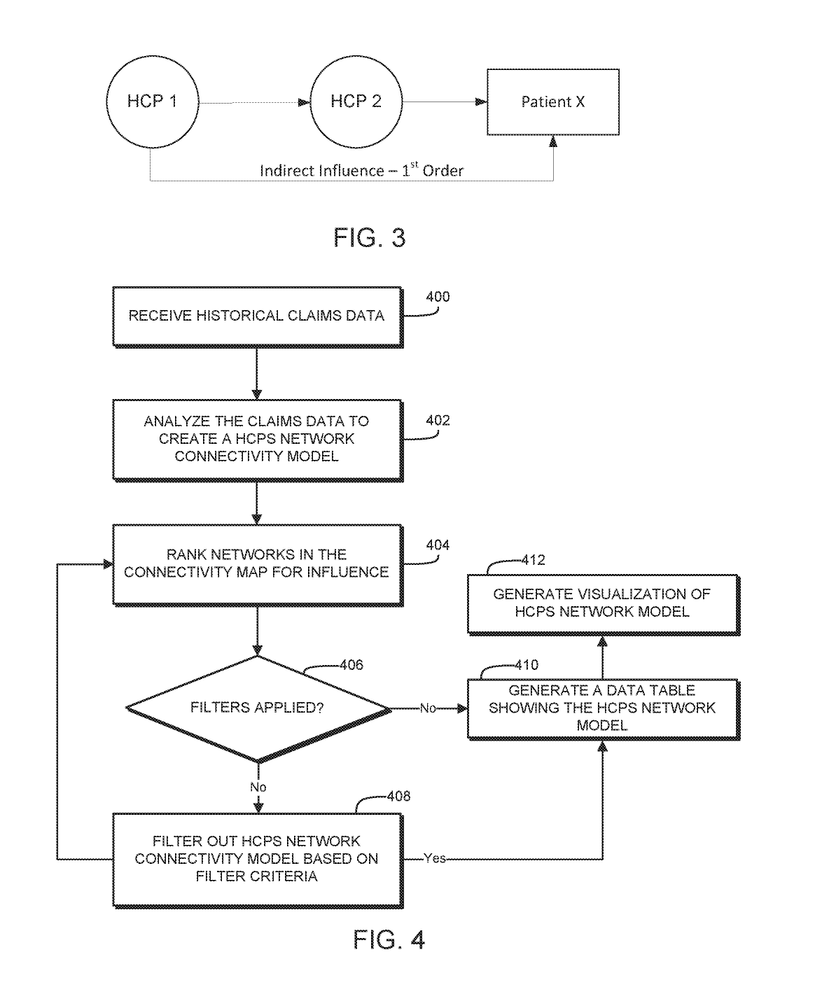

In the embodiment shown, the HCPs network analysis software 206 includes an influence ranking engine 213 that is configured to rank influence among HCPs, which may be built into the HCP network model 212. The term “influence” is meant to convey a relationship between a given Network and a Network Member, which is an HCP who is influenced by a specific Network. The influence rank is a number that is assigned to each Network that corresponds to the level of influence in a comparison to the other Networks in the cohort (e.g., 3=High, 2=Medium, and 1=Low). The specific method in which the influence rank is determined could be based on a variety of factors. For example, one factor in the influence rank could the total patients treated. Another factor could be the patients indirectly influenced by a HCP. The term “patients indirectly influenced” is intended to mean the patients treated by other HCPs who, in turn, are influenced by the selected HCP either directly or through other HCPs. There could be multiple orders of indirect influence. FIG. 3 shows an example of a 1^{st} order of indirect influence. In that example, HCP 1 referred Patient X to HCP 2, who treated Patient X. There could be other connections and influence if HCP 2, in turn, referred Patient X to another HCP and so forth. Other factors in the influence rank could be the total patients influences, which would be the sum of patients treated and patients indirectly influenced. The influence rank could also be based on HCPs indirectly influenced first order, which corresponds to the number of HCPs directly influenced by the Network HCP regardless of how many patients they treat. Another factor for the influence rank or score could be HCPs indirectly influenced which corresponds to the number of HCPs influenced by the Network HCP either directly or through other HCPs regardless of how many patients they treat. Yet another factor for the influence rank could be the Total HCPs influenced, which is the sum of all HCPs influenced indirectly.

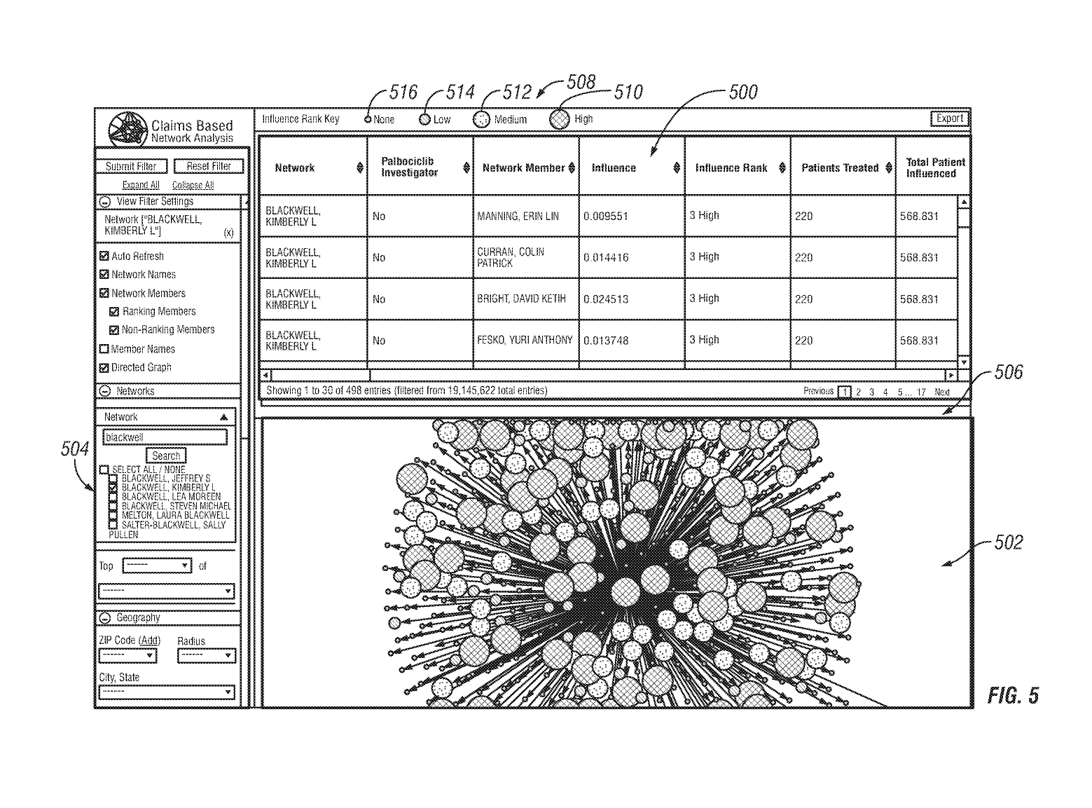

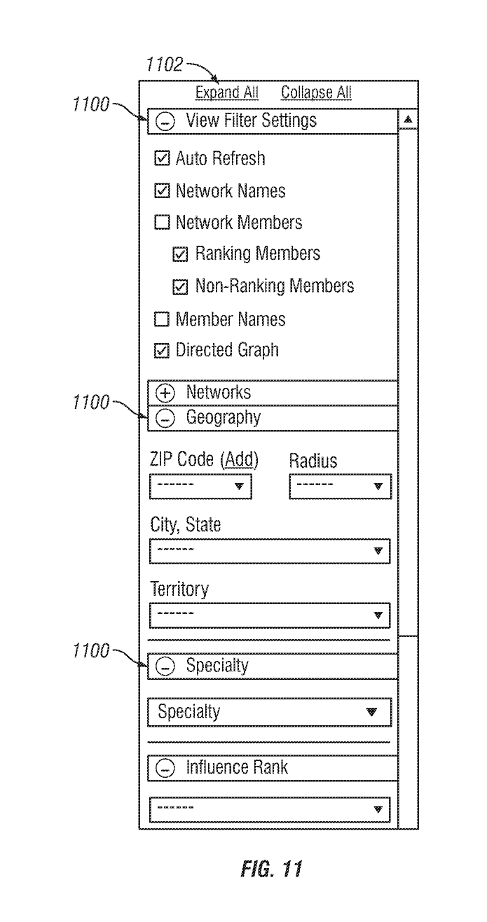

The HCPs network model 212 can be reviewed, analyzed and searched using an interface 214 to view various networks and identify influencers. In the embodiment shown, the interface 214 includes a data table 216, a graph area 218, and filter(s) 220. The data table includes a tabular form of data from the HCPs network model 212. The graph area 218 provides a visualization of the HCPs network model from which a user can graphically view the relative influence of different HCPs and connections between HCPs. The filter(s) 220 allow a user to search the HCPs network model 212 in a granulated manner using a variety of criteria, such as geographic regions, specialties, diseases, and/or specific health care providers. An example of the interface 214 is discussed below with respect to FIGS. 5-22.

FIG. 4 illustrates a method the computing device 100 could execute during use to create a HCPs network model and analyze the network model. The method starts with block 400 in which a program interacting with the claims data 208 stores the claims data in memory for analysis. The method advances to block 402 in which the claims data is analyzed by the connectivity mapping engine 210 to create a HCPs network connectivity model. Next, in block 404, the influence ranking engine 213 ranks the networks in the HCPs network model for influence such that an influence rank is assigned to each of the HCPs in the network model. Next, at block 406, a determination is made as to whether any filters need to be applied to the HCPs network model. If there are filter(s) to be applied, the method advances to block 408 and the HCPs network model is filtered based on the filter criteria; the method then advances to block 410. If there are no filter(s) to be applied, the method advances to block 410. At block 410, the interface displays a data table showing the HCPs network model. The method then advances to block 412 in which a visualization of the HCPs network model is generated and displayed.

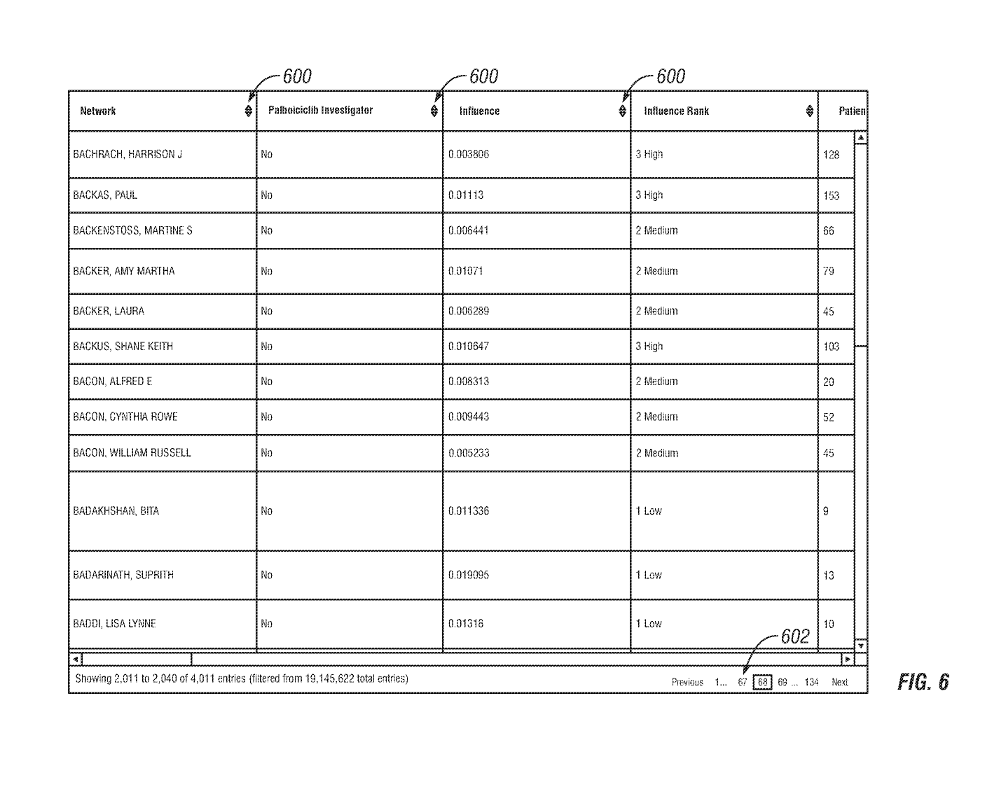

FIGS. 5-22 show an example interface through which the HCPs network model can be analyzed to, among other things, identify influencers. In the example shown, the interface 214 is divided into 3 basic regions: a data table 500, a graph area 502 and filters 504. The data table 500 and graph area 502 can be resized by dragging a bar 506 between them up and down to allow for a full screen data table or graph. In FIG. 6, a close up of the data table 500 is shown. The data table 500 is comprised of multiple columns, which can be sorted by ascending or descending order by clicking on the arrows 600 next to each column header. Page navigation 602 can be found at the bottom of the table in this example.

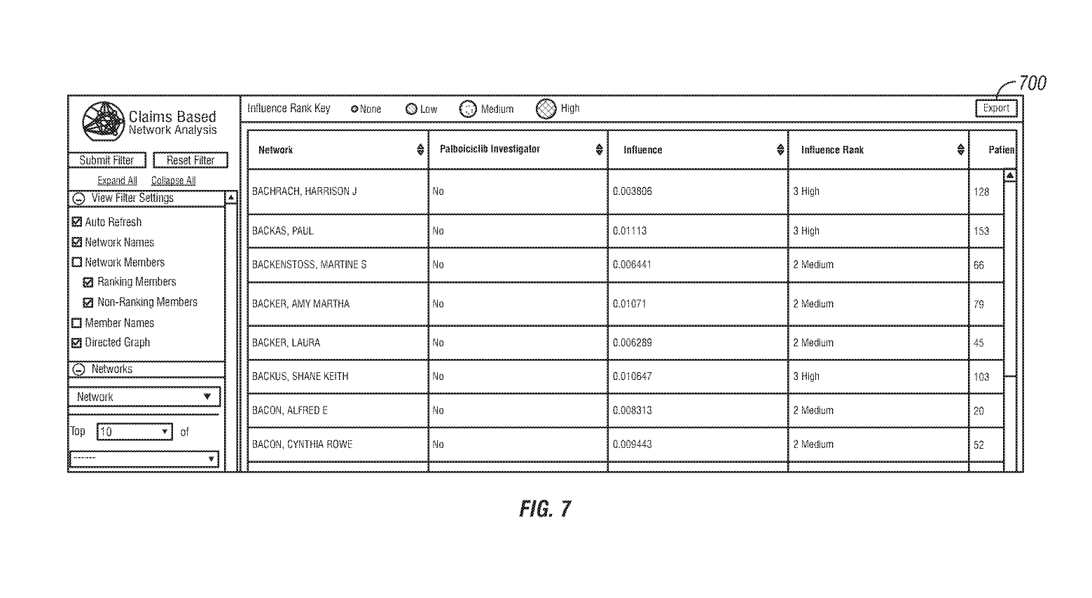

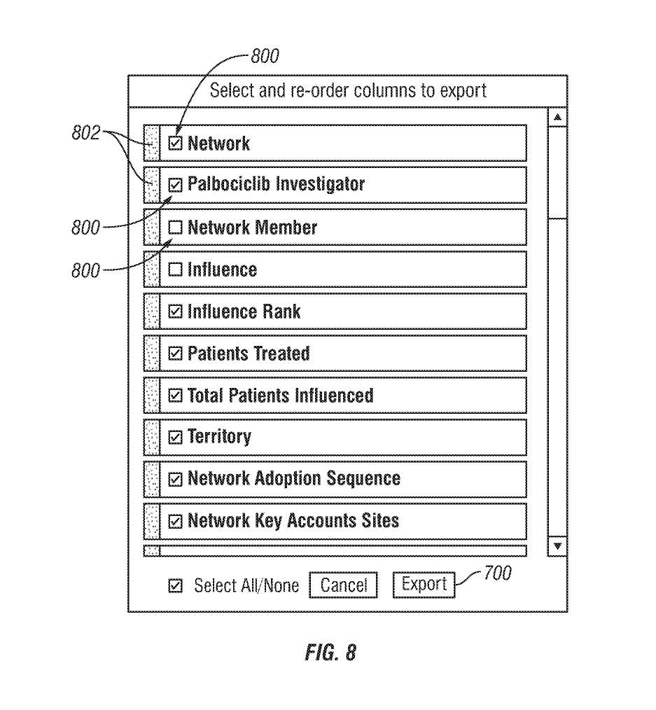

As best seen in FIG. 7, the data table 500 can be exported at any time by clicking on the export button 700, which in the top right corner in this example. Typically, it is recommended to filter down the data to a manageable set before exporting. FIG. 8 shows an example interface upon selecting export button 700 in which a pop-up window appears that allows the user to choose which columns to be exported by selecting/deselecting the check boxes 800. The user can also reorder the columns by clicking on the shaded area 802 next to the column name and dragging it to the desired position. The user would select the export button 700 upon making all selections.

Referring again to FIG. 5, in the embodiment shown a graph will appear once a dataset has been filtered down to preset number of rows, such as 1,000 rows or less. The graphs depict network connections, as well as influence rank. A key 508 for the graph can be found at the top of the screen in this example. The large circle 510 represents a high influencer, the smaller circle 512 represents a medium influencer, the smaller circle 514 represents a low influencer, and the dot 516 represents an HCP who is only an influencee and does not have any influence over other HCPs in the cohort. These distinctions could also be color-coded in the graph.

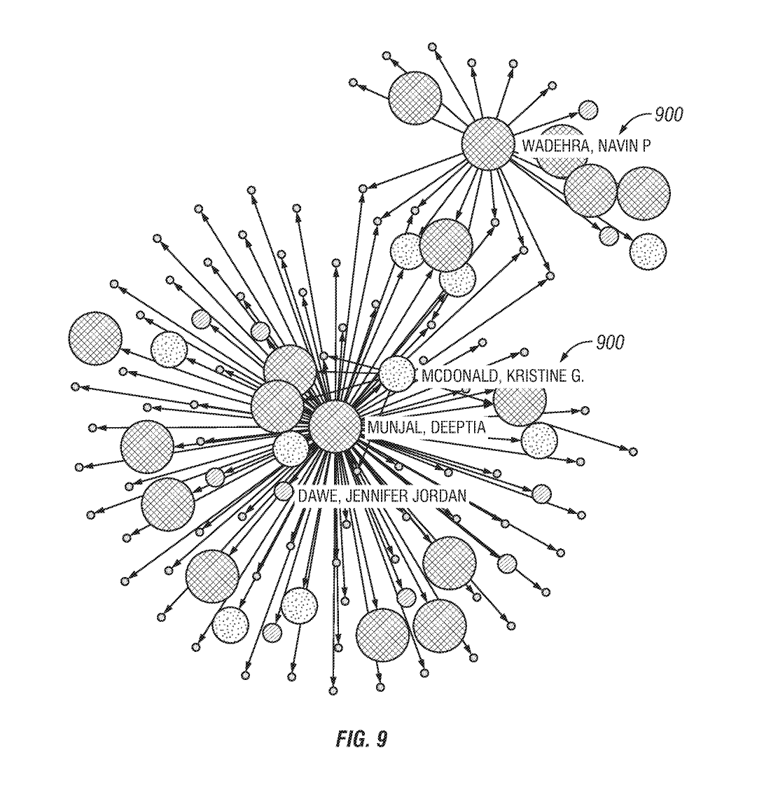

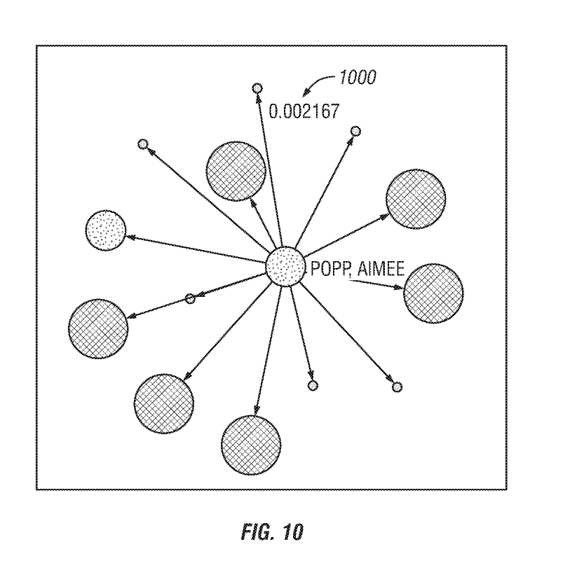

FIGS. 9 and 10 show certain interactions that the user may make with a graph. In the example of FIG. 9, hovering the user's mouse (or other input device) over any of the circles will display the name 900 of the HCP represented by the circle. In FIG. 10, hovering the user's mouse over any of the lines connecting HCPs 1000 will display the influence number between those two HCPs. In this example interface, double clicking on any of the network circles (also called nodes) in the graph will bring up that HCP's individual network in the graph, as well as the corresponding data grid.

In the example shown, the data filters are separated into 8 categories: Settings, Networks, Geography, Specialty, Influence Rank, Patients (Decile), HCPs (Decile), and Custom. The filter categories can be expanded or collapsed by selecting the circle 1100 next to the filter header. To expand or collapse all filters click on the corresponding button 1102 on the top of the filter section.

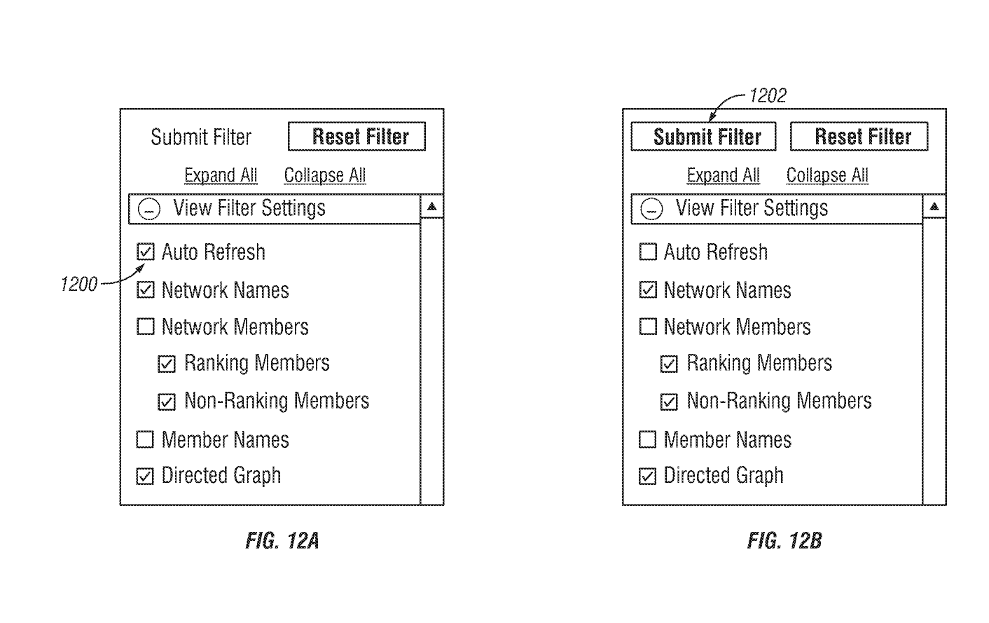

Referring to FIGS. 12A and 12B, this example interface includes an auto refresh 1200 will refresh the page with every filter selection or deselection. If the user is working with multiple filters at once it is best to turn off the Auto Refresh filter. When Auto Refresh is turned off, the user must select the Submit Filter button 1202 after selecting all desired filters.

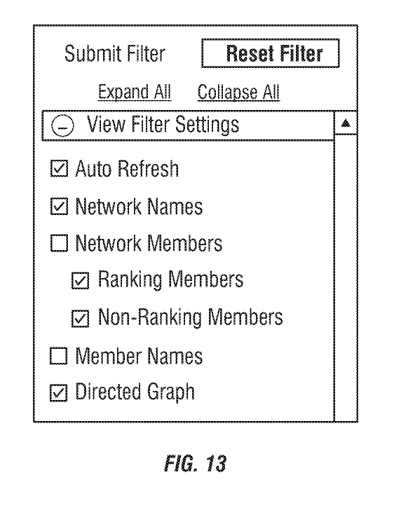

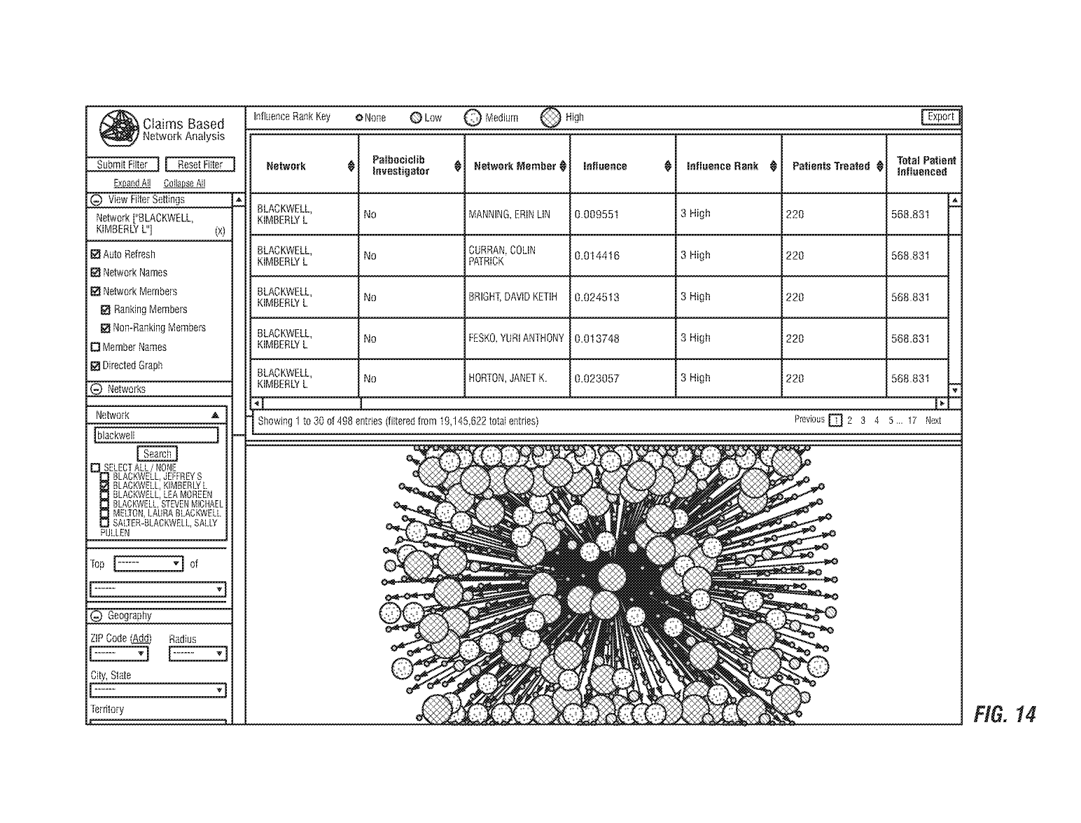

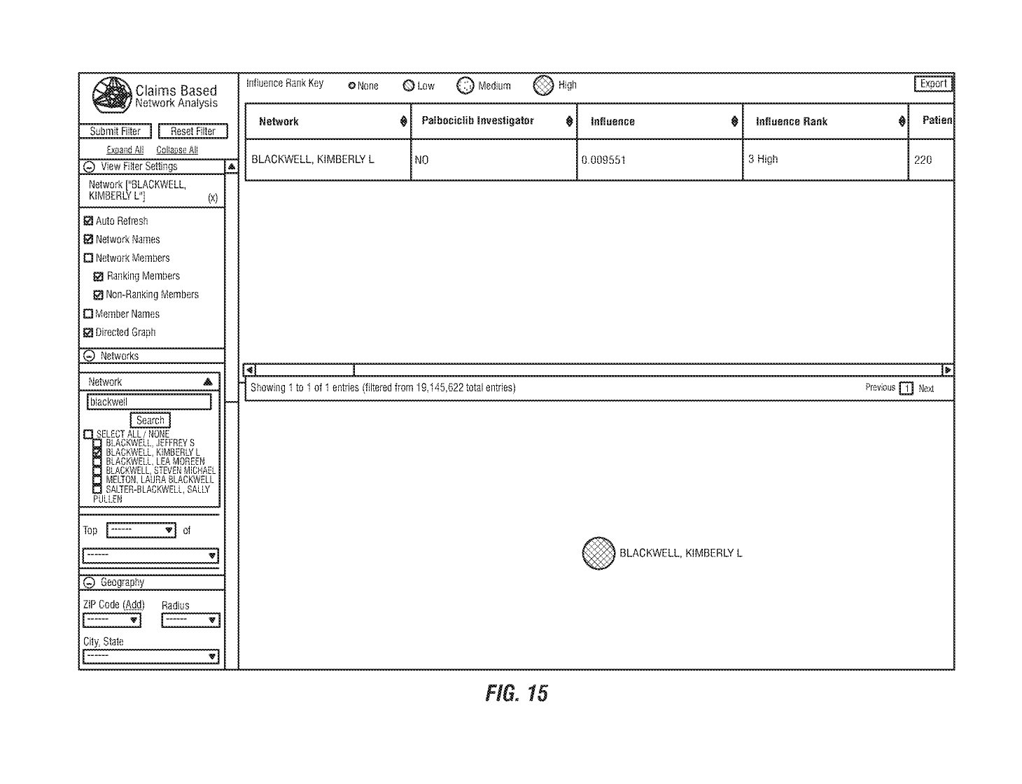

FIG. 13 shows an example network members filter. The Network Members filter gives the user the option to view only Network HCPs or to view the Network HCPs and their Network Members. The filter has two sub-categories: Ranking Members and Non-Ranking Members. Ranking Members are HCPs who have their own network themselves, while Non-Ranking Members are HCPs who are influencees only and do not have their own network. By selecting Network Members, this will show the user multiple rows of data in the data grid for each Network—one row for every connection as shown in FIG. 14. This filter will also show the user all connections in the graph. As shown in FIG. 15, deselecting Network Members will show the user only the Network names, but will not show any connections in both the data grid and the graph.

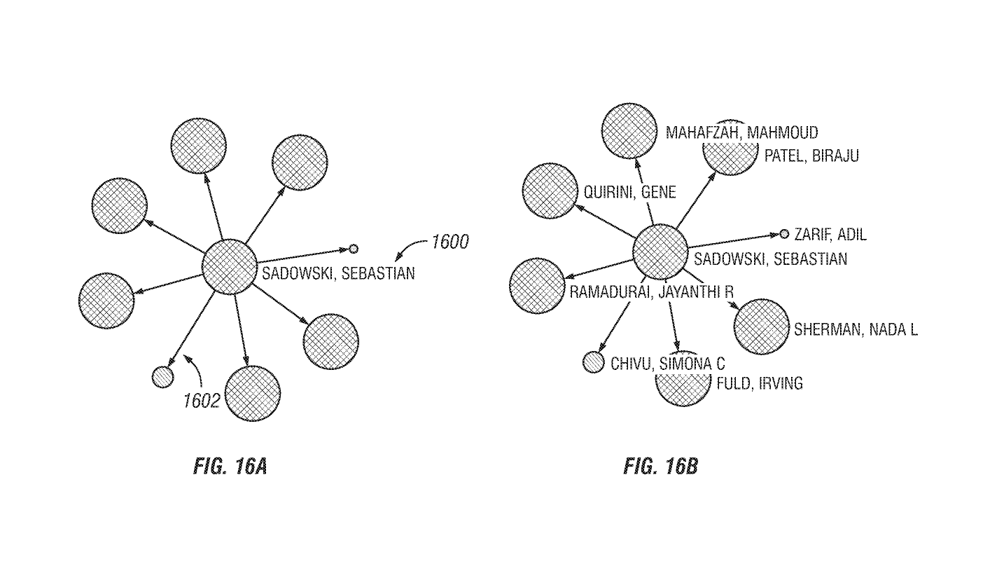

FIGS. 16A and 16B show example graph filters. Several filter settings apply directly to the graph. Selecting Network Names, 1600, this will display the name of the Network HCP the user is looking at as shown in FIG. 16A. Selecting member names will display the names of all Network Members on the graph as shown in FIG. 16B. Selecting the directed graph 1602 will display a directional arrow between connections.

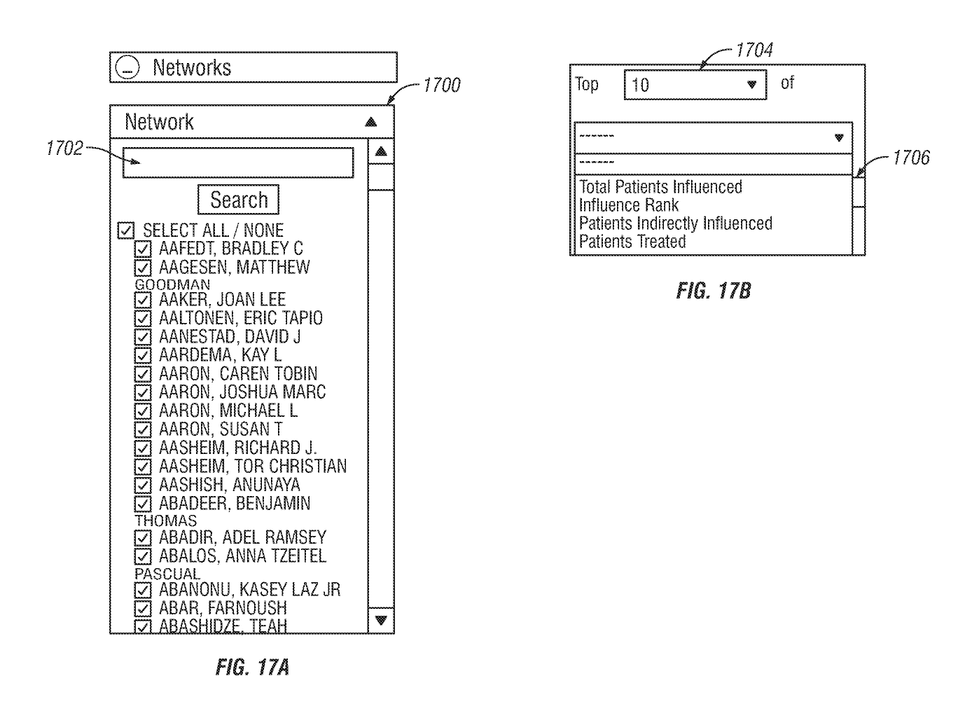

Referring to FIGS. 17A and 17B, the interface allows the user to search for a specific Network HCP by opening up the drop-down menu 1700 and either typing a name into the search bar 1702 or scrolling through the list to find the name the user is looking for. Multiple names can be selected at once. To filter top networks, the user would select a number from the dropdown 1704 and then select the category 1706 the user would like to filter by.

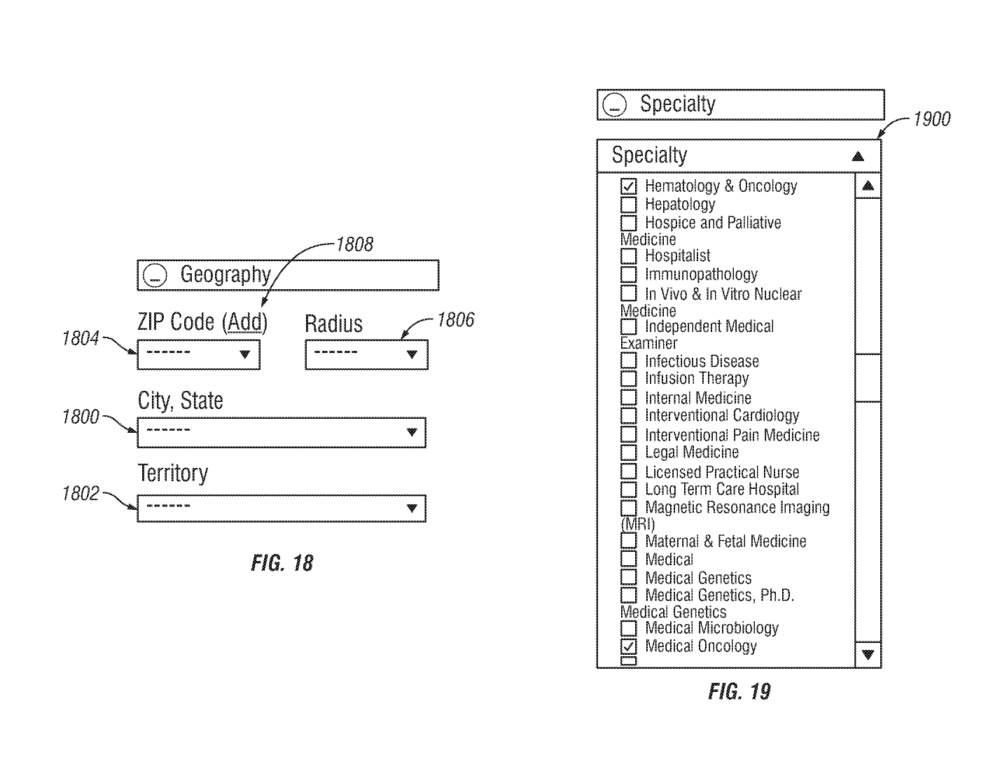

Referring now to FIG. 18, the interface could include geographic filters. For example, filters could be applied for City, State 1800; Territory 1802; and Zip Code 1804. After selecting a zip code the user could add a radius (listed in miles) 1806 to expand the search. The user could also add in a specific zip code 1808 that is not listed. FIG. 19 shows an example filter that allows a user to search for a specific specialty by opening up the dropdown menu 1900 and selecting one or more specialties.

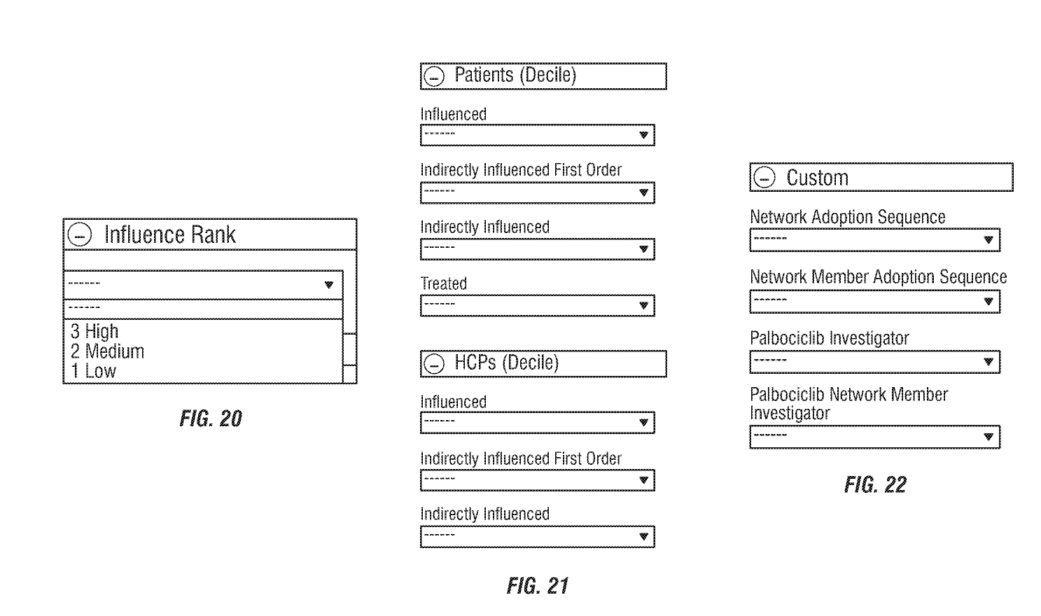

FIGS. 20-22 show an example interface in which HCPs can be filtered by influence (FIG. 20). Selecting a number will show results for that number and above (e.g., selecting 2 Medium will result in HCPs ranked 2 Medium or 3 High). HCPs can also be filtered by deciles in a variety of categories for patients (FIG. 21) and HCPs (FIG. 22). Selecting a number here will also show results for that number and above. In some cases, the interface could include custom filters, such as allowing the user to filter Networks and Network Members by Adoption Sequence (e.g., Early Adopters, Innovators) and by Investigators. In this example, the term “innovators” is intended to mean the first 2.5% of HCPs who begin prescribing a new medication after its launch. Innovators are characterized as HCPs who are willing to take risk and have the closest contact to researchers and other innovators. The term “early adopters” is intended to mean the next 13.5% of HCPs who prescribe a new medication after its launch. This group has the highest impact on all of the remaining adoption categories and tends to be more discrete in their choices than innovators. This characteristic gives Early Adopters more credibility with the Early Majority adopters who make up the largest segment of adopters.

Although the present disclosure has been described with reference to particular means, materials, and embodiments, from the foregoing description, one skilled in the art can easily ascertain the essential characteristics of the invention and various changes and modifications may be made to adapt the various uses and characteristics without departing from the spirit and scope of the invention.

What is claimed is:

1. An apparatus comprising:
a storage device; and
at least one processor coupled to the storage device, wherein the storage device stores a program for controlling the at least one processor, and wherein the at least one processor, being operative with the program, is configured to:
obtain historical claims data representative of encounters between a plurality of patients and health care providers (“HCPs”), including referrals between HCPs;
analyze the historical claims data to create a network model representative of connections between HCPs;
determine an influence score for the HCPs that represents a level of influence of a respective HCP in comparison to other HCPs that are connected to that respective HCP; and
transmit data representative of at least a portion of the network model with the influence score of those HCPs in that portion of the network.

2. The apparatus of claim 1, wherein at least a portion of the connections in the network model are based on referrals between HCPs.

3. The apparatus of claim 1, wherein the influence score is determined based at least in part on a number of patients treated directly by a HCP.

4. The apparatus of claim 1, wherein the influence score is determined based at least in part on patients of a selected HCP treated by other HCPs who are directly influenced by the selected HCP.

5. The apparatus of claim 4, wherein the influence score is determined based at least in part on patients of a selected HCP treated by other HCPs who are directly or indirectly influenced by the selected HCP.

6. The apparatus of claim 1, wherein the influence score is determined based at least in part on a sum of patients treated and patients indirectly influenced by a selected HCP.

7. The apparatus of claim 1, wherein the influence score is determined based at least in part on a number of HCPs influenced by a selected HCP either directly or indirectly regardless of how many patients treated by the selected HCP.

8. The apparatus of claim 1, wherein the program is configured to cause the processor to generate an interface from which the network model representative of connections between patients and HCPs can be viewed.

9. The apparatus of claim 8, wherein the interface includes a data table representative of one or more criteria that comprise the network model.

10. The apparatus of claim 8, wherein the interface includes a graphical visualization of the network model.

11. The apparatus of claim 10, wherein the graphical visualization includes one or more nodes representative of respective HCP and with connections between the nodes representing connected HCPs in the network model.

12. The apparatus of claim 10, wherein the graphical visualization is configured to visually differentiate nodes based on relative influence.

13. The apparatus of claim 12, wherein the visual differentiation between nodes based on relative influence is one or more of relative size and/or color of the nodes to identify relative influence between nodes.

14. The apparatus of claim 11, wherein responsive to detection of an input device hovering over a hovered node, the interface displays a name of the HCP corresponding to the hovered node.

15. The apparatus of claim 14, wherein responsive to selection of a selected node, the interface generates a network of the HCP corresponding to the selected node.

16. The apparatus of claim 11, wherein responsive to detection of an input device hovering over a hovered connection between nodes, the interface generates an influence score between the HCPs corresponding to the hovered connection.

17. The apparatus of claim 8, wherein the interface includes one or more filters configured to remove information of the network model from the interface based on user-selected filter criteria.

18. The apparatus of claim 17, wherein the one or more filters are configured to allow selection of one or more of the following as filter criteria: geographic regions, specialties, diseases, and/or specific health care providers.

19. A computer-implemented method, comprising:
obtaining historical claims data representative of encounters between a plurality of patients and health care providers (“HCPs”), including referrals between HCPs;
analyzing the historical claims data to create a network model representative of connections between HCPs;
determining an influence score for the HCPs that represents a level of influence of a respective HCP in comparison to other HCPs that are connected to that respective HCP; and
transmitting data representative of at least a portion of the network model with the influence score of those HCPs in that portion of the network.

20. The method of claim 19, wherein at least a portion of the connections in the network model are based on referrals between HCPs.

21. The method of claim 19, wherein the influence score is determined based at least in part on a number of patients treated directly by a HCP.

22. The method of claim 19, wherein the influence score is determined based at least in part on patients of a selected HCP treated by other HCPs who are directly influenced by the selected HCP.

23. The method of claim 22, wherein the influence score is determined based at least in part on patients of a selected HCP treated by other HCPs who are directly or indirectly influenced by the selected HCP.

24. The method of claim 19, wherein the influence score is determined based at least in part on a sum of patients treated and patients indirectly influenced by a selected HCP.

25. The method of claim 19, wherein the influence score is determined based at least in part on a number of HCPs influenced by a selected HCP either directly or indirectly regardless of how many patients treated by the selected HCP.

26. The method of claim 19, wherein the program is configured to cause the processor to generate an interface from which the network model representative of connections between patients and HCPs can be viewed.

27. The method of claim 26, wherein the interface includes a data table representative of one or more criteria that comprise the network model.

28. The method of claim 26, wherein the interface includes a graphical visualization of the network model.

29. The method of claim 28, wherein the graphical visualization includes one or more nodes representative of respective HCP and with connections between the nodes representing connected HCPs in the network model.

30. The method of claim 28, wherein the graphical visualization is configured to visually differentiate nodes based on relative influence.

31. The method of claim 30, wherein the visual differentiation between nodes based on relative influence is one or more of relative size and/or color of the nodes to identify relative influence between nodes.

32. The method of claim 29, wherein responsive to detection of an input device hovering over a hovered node, the interface displays a name of the HCP corresponding to the hovered node.

33. The method of claim 30, wherein responsive to selection of a selected node, the interface generates a network of the HCP corresponding to the selected node.

34. The method of claim 29, wherein responsive to detection of an input device hovering over a hovered connection between nodes, the interface generates an influence score between the HCPs corresponding to the hovered connection.

35. The method of claim 29, wherein the interface includes one or more filters configured to remove information of the network model from the interface based on user-selected filter criteria.

36. The method of claim 33, wherein the one or more filters are configured to allow selection of one or more of the following as filter criteria: geographic regions, specialties, diseases, and/or specific health care providers.

37. A tangible, non-transitory computer readable medium storing instructions that, when executed by at least one processor, causes the at least one processor to perform a method comprising:
obtaining historical claims data representative of encounters between a plurality of patients and health care providers (“HCPs”), including referrals between HCPs;
analyzing the historical claims data to create a network model representative of connections between HCPs;
determining an influence score for the HCPs that represents a level of influence of a respective HCP in comparison to other HCPs that are connected to that respective HCP; and
transmitting data representative of at least a portion of the network model with the influence score of those HCPs in that portion of the network.
